(19) United States
(12) Patent Application Publication
(10) Pub. No.: US 2025/0305982 A1
(43) Pub. Date: Oct. 2, 2025

(54) DEVICE AND METHOD FOR THE DETECTION OF BIOMARKERS ASSOCIATED WITH NEURODEGENERATIVE DISEASES

(71) Applicants: CONSEJO NACIONAL DE INVESTIGACIONES CIENTÍFICAS Y TÉCNICAS (CONICET), Ciudad Autónoma de Buenos Aires (AR); UNIVERSIDAD NACIONAL DE TUCUMÁN, San Miguel de Tucumán, Tucumán (AR); SISTEMA PROVINCIAL DE SALUD DE TUCUMÁN, San Miguel de Tucumán, Tucumán (AR); SKYBIO LLC, Miami, FL (US); UNIVERSIDAD DE BUENOS AIRES, Ciudad Autónoma de Buenos Aires (AR)

(72) Inventors: Oscar José VARELA, CABA Buenos Aires (AR); Rosana Nieves CHEHÍN, Yerba Buena (AR); Cesar Luis ÁVILA, San Miguel de Tucumán (AR); Sergio Benjamín SOCÍAS, Yerba Buena (AR); Diego PLOPER, San Miguel de Tucumán (AR); Esteban VERA PINGITORE, Yerba Buena (AR); Martín LUONG, CABA Buenos Aires (AR); Verónica Elena MANZANO, CABA Buenos Aires (AR); Rossana Elena MADRID, Tucumán (AR); Adriana Andrea KOLENDER, Ciudad Autónoma de Buenos Aires (AR); María Belén MACHÍN, Tucumán (AR); Agustín Osvaldo PERNICONE, Ciudad Autónoma de Buenos Aires (AR); Silvana Estefanía SOLIZ SANTANDER, Tucumán (AR)

(73) Assignees: CONSEJO NACIONAL DE INVESTIGACIONES CIENTÍFICAS Y TÉCNICAS (CONICET), Ciudad Autónoma de Buenos Aires (AR); UNIVERSIDAD NACIONAL DE TUCUMÁN, San Miguel de Tucumán, Tucumán (AR); SISTEMA PROVINCIAL DE SALUD DE TUCUMÁN, San Miguel de Tucumán, Tucumán (AR); SKYBIO LLC, Miami, FL (US); UNIVERSIDAD DE BUENOS AIRES, Ciudad Autónoma de Buenos Aires (AR)

(21) Appl. No.: 18/863,290

(22) PCT Filed: May 5, 2023
(86) PCT No.: PCT/IB2023/054715

§ 371 Date: Nov. 5, 2024

Related U.S. Application Data
(60) Provisional application No. 63/339,220, filed on May 6, 2022.

(51) Int. Cl. G01N 27/327; G01N 27/02; G01N 33/543; G01N 33/68

(52) U.S. Cl. CPC G01N 27/3277; G01N 27/026; G01N 33/54386; G01N 33/6896; G01N 2333/4709; G01N 2800/28

(57) ABSTRACT

The present disclosure provides devices for the detection and/or quantification of neurotoxic amyloid-type protein aggregates, comprising a doxycycline derivative immobilized on an appropriate surface, as well as electrochemical and immunochemical methods associated to the use of such devices.

### FIELD OF THE INVENTION

The present invention relates to the detection of biomarkers for the diagnosis of neurodegenerative diseases, in particular synucleinopathies, such as Parkinson's disease and tauopathies, such as Alzheimer's disease. More particularly, the invention relates to devices for the detection of said biomarkers, comprising a doxycycline derivative immobilized on an appropriate surface, as well as to electrochemical and immunochemical methods associated to the use of such devices.

### BACKGROUND OF THE INVENTION

The ratio of elderly population is growing at a global scale. According to the World Health Organization (WHO), the percentage of people above 60 years old is expected to double between 2015 and 2050 across the world. Taking into account that elderly people are more likely to suffer neurodegenerative diseases, the effective diagnosis and treatment of said diseases is increasingly important. Among the several existing age-related neurodegenerative diseases, Parkinson's disease (PD) and Alzheimer's disease are prevalent.

According to the World Health Organization (WHO), the prevalence of PD has doubled in the last 25 years, reaching more than 10 million people in 2020. However, these figures may be underestimated due to the lack of a standardized global registration system and the variability in diagnostic criteria used in different countries. Aging is considered the main risk factor for the disease, and the increase in life expectancy predicts a strong increase in age-related diseases in the coming decades. However, the WHO also warns that the prevalence of PD is increasing at a faster rate than other ND, and projections predict a doubling of the number of cases in the coming decades. On the other hand, the economic burden of symptomatic treatments is enormous, not only for patients and family members, but also for healthcare systems worldwide. In the USA, for example, in 2017, this figure was estimated at 51 billion USD, and projections based on the increasing incidence of the disease threaten to strain healthcare systems worldwide in the coming decades. These figures have mobilized the scientific community to study different mechanisms capable of stopping the neurogenerative process of the disease.

However, the efficiency of any neuroprotective treatment is subject to its administration at very early stages of the disease, which must be diagnosed before the emergence of the motor symptoms typically associated with PD.

Despite the vast amount of effort invested in quantifying alpha-synuclein (AS) aggregates, which are considered the main biomarkers for PD, to this date there are no biochemical analyses for biological fluids capable of detecting the first stages of the disease.

The existing techniques for diagnosing and/or monitoring PD, mainly from cerebrospinal fluid (CSF) samples, are detailed below:
Conventional ELISA (enzyme-linked immunosorbent assay).
This immunoassay involves immobilizing anti-synuclein antibodies on a polymeric plaque. The main disadvantage of this technique is that the aggregated species of AS are very heterogeneous, while antibodies are sensitive to the conformation of their intended antigen. Therefore, they are only capable of detecting some types of aggregates thus failing to embody the great diversity of existing species. This renders this technique unspecific, and potentially affects its sensitivity (Ganguly et al. (2021); Eusebi et al. (2016); Atik et al. (2016)).
AS seed amplification also named real-time quaking-induced conversion (RT-QuIC) or AS seed amplification assays (SAAs).
Based on the potential of this technique to differentiate people with Parkinson's disease from healthy controls, a large cross-sectional analysis (1123 participants) using SAA show that the assay classifies people with Parkinson's disease with high sensitivity and specificity, provides information about molecular heterogeneity, and can also detect prodromal individuals before diagnosis (Siderowf et al. (2023).
The main limitation of RT-QuIC is the availability of recombinant human AS, whose market cost renders the massive application of this technique complicated. It also required specialized equipment not available in low and medium complexity laboratories, and human resources training for its implementation. Additionally, there is no evidence to this date suggesting it might be miniaturized for its application as a point-of-care (POC) system. (Luan, M., et al. (2022); Poggiolini I, et al. (2022); Kuzkina, A, et al. (2021)).
Nuclear imaging techniques:
   Single-photon emission computed tomography (SPECT)
   This technique uses a radioligand that binds to dopamine receptors detecting the emitted gamma radiation. SPECT can provide information about the functioning of dopaminergic neurons typically associated with Parkinson's disease.
   Positron emission tomography (PET)
   PET uses a radioactive tracer that emits positrons, and can also be used to assess dopamine activity in the brain by using radioligands that bind to dopamine receptors or transporters.
The nuclear image techniques have limited sensitivity and are not effective in detecting subtle changes in dopamine levels in the brain in the early stages of the disease, which could result in false-negative results. In addition, the cost of this technology limits its availability in healthcare system with low or medium complexity. Eventually, interpreting results can be complex and requires expertise in nuclear medicine.
In conclusion, these techniques are usually reserved for a diagnostic confirmation of PD in the motor phase (Golan H, et al. (2022); Pavese, N, et al. (2011); Moore, R. Y., et al. (2008)).
Immunomagnetic reduction by means of a superconducting quantum interference device (IMR-SQUID).
This technique uses antibody-functionalized magnetic nanoparticles and a high-sensitivity SQUID magnetometer. Even though the technique is promising because it detects the protein at a femtomolar level, its massive application would be complicated due to the high cost of the equipment, and due to the difficulty exhibited by antibodies to detect different aggregated species (Yang, S Y., et al. (2016)).

Jang et al. (2020) disclose an electrochemical sensor for detecting AS oligomers for an early PD diagnosis. The sensor comprises a methylene blue-aptamer adsorbed on an electrode, which is desorbed when bound to an AS oligomer present in a sample, thus generating a detectable variation in the electrical signal provided by the electrode. The electrode used in the sensor is a reduced graphene oxide electrode.

However, there is a need for alternative AS aggregates-detection techniques with low operational costs, using easily accessible equipment, and with a broad specificity to facilitate the detection of several types of aggregates potentially present in a sample.

Regarding AD, it is a devastating neurological condition that gradually erodes memory, cognitive abilities, and behavior. By far, AD is the most common form of dementia; being more prevalent than vascular dementia, mixed dementia, Lewy body dementia (LBD) and frontotemporal dementia (FTD). Currently, AD accounts for 60-80% of all cases of dementia. Likewise, the prevalence of AD is only expected to rise with time. According to the Centers for Disease Control and Prevention (CDC), approximately 5.8 million Americans currently suffer from AD, and this figure is projected to increase as the population ages. As a matter of fact, beyond the age of 65, the risk of developing AD doubles every 5 years. The escalating prevalence of the disease is a significant burden on healthcare systems, as the demand for care continues to rise as the disease progresses. This is made even more challenging by the fact that death rates for AD are on the rise, in contrast to heart disease and cancer, whose death rates are declining. The cost of caring for AD patients is also alarmingly high, estimated at over $500 billion annually, a figure that is expected to increase as the population ages.

A key neuropathological characteristic of AD is the presence in the brain of deposits of the microtubule-associated protein tau (Tau), called neurofibrillary tangles (NFTs), in various morphologies, which appear many years before the onset of clinical symptoms. While the accumulation of NFTs was first described in AD, it is worth noting that other neurodegenerative disorders, such as frontotemporal dementia, Pick's disease, progressive supranuclear palsy, and corticobasal degeneration, have also been associated with the presence of NFTs. In fact, some of the strongest evidence supporting Tau's involvement in neurodegenerative diseases comes from the identification of mutations in patients with frontotemporal dementia, which highlights the potential of Tau to be a causative factor in the development of these types of diseases.

Tau pathology can spread from one region of the brain to another, similar to prions. Growing evidence demonstrates that aggregated species of Tau spread along neuroanatomically connected brain areas through a “prion-like” mechanism, transferring abnormal Tau seeds from donor to a recipient cell, thus generating new Tau seeds. Therefore, early diagnosis of AD is essential because it provides the opportunity for a pharmacological neuroprotective intervention to slow down the progression of the disease. In preclinical stages of Alzheimer's disease, there may be no noticeable symptoms, but changes in the brain are already occurring, such as the accumulation of amyloid β (Aβ) and Tau proteins. These changes may be detectable through imaging and biomarker detection and quantification.

Ongoing research in biomarker detection for AD is currently focused on the development of reliable and sensitive assays for detecting biomarkers in bodily fluids such as cerebrospinal fluid (CSF), blood plasma, and urine. Additionally, there is a concerted effort to improving imaging techniques to detect biomarkers other than Aβ aggregates in the brain by neuroimaging.

Tau aggregates comprise a wide variety of species in a dynamic process until they are recruited in the NFTs. Most of them have been involved in cellular toxicity. Furthermore, there is growing evidence suggesting that Tau aggregates have a crucial role in spreading the pathology from one neuron to another, triggering the aggregation process in healthy neurons. These features render Tau aggregates widely recognized as a robust biomarker, but their detection remains challenging due to the absence of an antibody capable of detecting all species. Classical immunological detection methods that rely solely on epitope/paratope interactions are inadequate in recognizing all conformational varieties of aggregates, frequently resulting in false positives or false negatives.

While all amyloid aggregates, including those formed by Tau, share a cross-β structure, the compactness of this structure can either hide epitopes or expose new ones. As a result, developing antibodies capable of specifically recognizing and binding to amyloid aggregates has proven to be a true challenge. In contrast, small molecules like Thioflavin T (ThT), Congo red (CR), and Doxycycline show strong and specific binding to amyloid aggregates structures (González-Lizárraga (2017), Medina (2021)).

As for PD, there are several existing techniques for diagnosing and monitoring AD, both in low and medium complexity laboratories, as follows:

### Immunohistochemistry

Immunohistochemistry is used to confirm a diagnosis of Alzheimer's disease after death by examining the presence and distribution of Tau aggregates in the brain tissue samples obtained during autopsy. This technique involves staining thin sections of brain tissue with specific antibodies that bind to Tau protein. The antibodies are labeled with a color-producing molecule that enables visualization of the Tau aggregates under a microscope.

### Immunofluorescence

Immunofluorescence is a technique that is similar to immunohistochemistry, but it uses fluorescent dyes instead of color-producing molecules to label Tau. The technique involves incubating brain tissue samples with antibodies that specifically bind to Tau, which are conjugated with a fluorescent dye. It's worth noting that while immunofluorescence is a valuable tool in Alzheimer's disease research, it is not routinely used for clinical diagnosis during autopsy.

### Conventional ELISA

Conventional ELISA is limited in its ability to detect Tau aggregated species due to its reliance on a single monoclonal capture antibody. This limitation reduces its capability to detect the variety of Tau aggregate species. Commonly used commercial ELISA tests, such as BioLegend, Abcam, Thermofisher, and IBL International, are designed to detect total Tau protein and phosphorylated Tau as biomarkers. Unfortunately, these tests suffer from decreased sensitivity and specificity, resulting in false positives or negatives results (Mounsey (2018)). This highlights the need for better capture molecules to enhance the accuracy of Tau aggregate detection.

### PET Imaging

This technique is a powerful tool for visualizing and quantifying the accumulation of pathological proteins in the brains of patients with Alzheimer's disease (AD). Several radiotracers have been developed to target Tau aggregates in the brain such as [¹⁸F]Tau PET tracers. As discussed above for PD, this diagnostic technique is available mostly in high complexity medical centers and research institutions due to the cost of the equipment and the radiotracers. Additionally, there are limitations to the interpretation of PET imaging, as it can only provide information on the distribution and density of pathological proteins and not on their specific composition or location within the brain. Moreover, the interpretation of PET results can be challenging, as there is no standardized method for image analysis and quantification.
   Single-Molecule Fluorescence Resonance Energy Transfer (smFRET)
This is a biophysical technique that can be used to measure intramolecular distances in proteins, including Tau. This technique has been used to investigate conformational changes in Tau with or without the presence of aggregation inducers, providing insight into the mechanisms underlying Tau aggregation in AD. However, smFRET is a sophisticated technique that requires specialized equipment and expertise and is typically only available through service providers or in specialized research laboratories.

### RT-QuIC

This technology has been explored as a diagnostic tool for AD. It involves the detection of misfolded proteins, such as Tau and beta-amyloid, in CSF or other biological samples by inducing conversion of normal proteins into abnormal aggregates that can be detected through changes in fluorescence or turbidity. RT-QuIC has shown promising results in detecting Tau aggregates in CSF samples from AD patients with high sensitivity and specificity. However, it is still in the experimental stage and not yet widely available for clinical use. It also requires specialized equipment and expertise, which limits its use to more sophisticated laboratories.

It is evident that there is still a need for alternative techniques that allow the detection of biomarkers associated with AD with low operational costs, using easily accessible equipment, and with a broad specificity to facilitate the detection of several types of aggregates potentially present in a sample.

Moreover, it would be highly desirable to develop a versatile technique, suitable for detecting biomarkers associated with neurodegenerative diseases at an early stage. Particularly, it would be very useful to obtain a device and a method for detecting biomarkers associated to diseases such as PD and AD.

Doxycycline is a long-known antibiotic of the tetracycline family, which has been used for treating a broad range of bacterial infections, such as bacterial pneumonia, cholera, syphilis, among many others.

### SUMMARY OF THE INVENTION

According to a first aspect, the present invention provides a device for detecting neurotoxic amyloid-type protein aggregates, comprising a doxycycline derivative capable of binding to neurotoxic amyloid-type protein aggregates, wherein the doxycycline derivative is immobilized on a surface.

In an embodiment of this aspect of the invention, the device comprises a doxycycline derivative of formula (I):

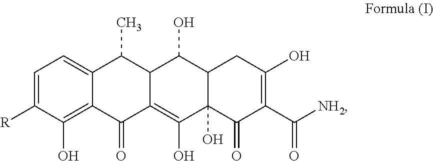

immobilized on a surface, wherein R is selected from the group consisting of:
i—a substituent selected from the group consisting of H, NH₂, COOH, (CH₂)ₙX, COOCH₂(CH₂)ₙX, wherein n=0-10 and X is selected from Cl, Br and I;
ii—an alkyl or aryl linker with a thiol end group;
iii—

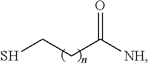

wherein n=0-10;
iv—

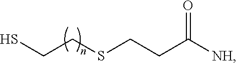

wherein n=0-10; and
v—

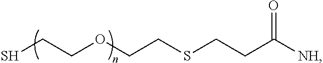

wherein n=0-10.

In a preferred embodiment, R is a substituent selected from the group consisting of H, NH₂, COOH, (CH₂)ₙX, COOCH₂(CH₂)ₙX, wherein n=0-10 and X is selected from Cl, Br and I.

In a particularly preferred embodiment of the invention, the device comprises a doxycycline derivative of formula (II),

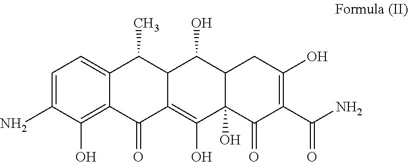 [SMILES: C[C@H]1c2ccc(N)c(O)c2C(=O)C2=C(O)[C@]3(O)C(=O)C(C(N)=O)=C(O)CC3[C@@H](O)C21]

immobilized on a surface.

In another embodiment of this aspect of the invention, the surface is selected from a polymeric surface and a functionalized metallic surface.

In a specific embodiment of the invention, the surface is a polymeric surface, wherein the polymeric surface comprises a polymer selected from the group consisting of polystyrene, a polystyrene/divinylbenzene copolymer, and other synthetic or natural polymers where doxycycline derivatives may be immobilized. Preferably, the polymeric surface comprises polystyrene.

In a preferred embodiment of the invention, the doxycycline derivative is immobilized on the polymeric surface by being covalently bound through a linker to a blocking agent adsorbed on the surface. Preferably, the blocking agent adsorbed on the polymeric surface is bovine serum albumin.

In a particularly preferred embodiment, the device comprises the doxycycline derivative of formula (II) immobilized on a polymeric surface by being covalently bound through glutaraldehyde to bovine serum albumin adsorbed on the surface.

In another specific embodiment of the invention, the surface is a functionalized metallic surface. Preferably, the functionalized metallic surface is a functionalized gold surface. More preferably, the functionalized gold surface comprises a self-assembled monolayer (SAM) of a mercapto acid. Most preferably, the functionalized gold surface comprises a SAM of 3-mercaptopropionic acid.

In a particularly preferred embodiment, the device comprises the doxycycline derivative of formula (II) immobilized on a functionalized metallic surface, wherein the functionalized metallic surface is a functionalized gold surface, wherein the functionalized gold surface comprises a SAM of 3-mercaptopropionic acid, and wherein the doxycycline derivative of formula (II) is covalently bound to the SAM of 3-mercaptopropionic acid.

It is another aspect of the present invention to provide an in vitro method for detecting and/or quantifying neurotoxic amyloid-type protein aggregates, comprising:
i—providing a device for detecting neurotoxic amyloid-type protein aggregates according to the first aspect of the invention;
ii—contacting the device with a sample in which the presence and/or concentration of said neurotoxic amyloid-type protein aggregates is to be determined; and
iii—determining the presence and/or concentration of said neurotoxic amyloid-type protein aggregates by a detection technique.

In an embodiment of this aspect, the neurotoxic amyloid-type protein aggregates to be detected and/or quantified are alpha-synuclein (AS) aggregates.

In another embodiment of this aspect, the neurotoxic amyloid-type protein aggregates to be detected and/or quantified are tau protein (Tau) aggregates.

In an embodiment of this aspect of the invention, the detection technique is selected from the group consisting of an immunochemical assay and an electrochemical assay.

In a particular embodiment of this aspect of the invention, the detection technique of step iii—is an electrochemical assay. Preferably, the electrochemical assay uses a technique selected from the group consisting of cyclic voltammetry (CV) and electrochemical impedance spectroscopy (EIS) for detecting the neurotoxic amyloid-type protein aggregates. More preferably, the electrochemical assay uses cyclic voltammetry for detecting the neurotoxic amyloid-type protein aggregates.

In another particular embodiment of this aspect of the invention, the detection technique of step iii—is an immunochemical assay, and step iii—comprises the sub-steps of:
a) contacting the device with an antibody appropriate for binding to the neurotoxic amyloid-type protein aggregates to be detected and/or quantified; and
b) measuring the bound antibody by a measuring technique.

In a preferred embodiment of this aspect of the invention, the neurotoxic amyloid-type protein aggregates to be detected and/or quantified are AS aggregates, and the method comprises:
i—providing a device for detecting neurotoxic amyloid-type protein aggregates according to the first aspect of the invention;
ii—contacting the device with a sample in which the presence and/or concentration of said AS aggregates is to be determined; and
iii—determining the presence and/or concentration of said AS aggregates by an immunochemical assay, wherein the immunochemical assay further comprises:
   a) contacting the device with an antibody appropriate for binding to AS aggregates; and
   b) measuring the bound antibody by a measuring technique.

In another preferred embodiment of this aspect of the invention, the neurotoxic amyloid-type protein aggregates to be detected and/or quantified are Tau aggregates, and the method comprises:
i—providing a device for detecting neurotoxic amyloid-type protein aggregates according to the first aspect of the invention;
ii—contacting the device with a sample in which the presence and/or concentration of said Tau aggregates is to be determined; and
iii—determining the presence and/or concentration of said Tau aggregates by an immunochemical assay, wherein the immunochemical assay further comprises:
   a) contacting the device with an antibody appropriate for binding to Tau aggregates; and
   b) measuring the bound antibody by a measuring technique.

It is yet another aspect of the invention to provide a method for preparing a device for detecting neurotoxic amyloid-type protein aggregates according to the invention, comprising:
i—providing a surface capable of immobilizing a doxycycline derivative; and
ii—contacting the surface able for immobilizing a doxycycline derivative with a solution comprising a doxycycline derivative capable of binding to neurotoxic amyloid-type protein aggregates in conditions such that the doxycycline derivative capable of binding to neurotoxic amyloid-type protein aggregates is immobilized on the surface.

In an embodiment of this aspect of the invention, the surface capable of immobilizing a doxycycline derivative is selected from the group consisting of a polymeric surface and a functionalized metallic surface.

In a specific embodiment of this aspect of the invention, the surface is a polymeric surface, wherein the polymeric plastic surface comprises a polymer selected from the group consisting of polystyrene, a polystyrene/divinylbenzene copolymer, and other synthetic or natural polymers where doxycycline and derivatives thereof may be immobilized. Preferably, the polymeric plastic surface comprises polystyrene.

In an embodiment of this aspect of the invention, the surface is a polymeric surface, and step i of the method further comprises the following sub-steps:
a) contacting the polymeric surface with a solution comprising a blocking agent so as to cause the adsorption of said blocking agent onto the polymeric surface, thus obtaining a blocked polymeric surface; and
b) contacting the blocked polymeric surface with a solution comprising a linker to obtain a polymeric surface capable of immobilizing a doxycycline derivative.

Preferably, the blocking agent is bovine serum albumin, and the linker is glutaraldehyde.

In another specific embodiment of this aspect of the invention, the surface is a functionalized metallic surface, and the method further comprises a step prior to step i, wherein a metallic surface is contacted with a solution comprising a functionalizing agent to obtain said functionalized metallic surface. Preferably, the metallic surface is a gold surface. Also preferably, the functionalizing agent is a mercapto acid. More preferably, the functionalizing agent is 3-mercaptopropionic acid.

It is yet another aspect of the invention to provide a kit for detecting and quantifying biomarkers associated with PD, comprising
i—a polymeric surface functionalized or ready to be functionalized with a doxycycline derivative according to the invention; and
ii—detection reagents.

It is yet another aspect of the invention to provide a point-of-care (POC) biosensor for detecting biomarkers associated with PD, comprising an electrode functionalized or ready to be functionalized with a doxycycline derivative according to the invention.

### BRIEF DESCRIPTION OF THE DRAWINGS

The following figures form part of the present specification and are included to further illustrate certain aspects of the present invention.

FIG. 1. CV characterization of each different stage of modification of a gold surface during the manufacture of a device according to the invention. Doxy-5 refers to the compound of formula (II) according to the invention.

FIG. 2. Zoom on the oxidation peak of: A) the cyclic voltammogram obtained for different AS concentrations with a gold-based device according to the invention; and B) the cyclic voltammogram of the control condition without the addition of Doxy-5.

FIG. 3. Evaluation of the concentration of the compound of formula (II) for well sensitization in a polystyrene-based device according to the invention. Doxy-5 refers to the compound of formula (II) according to the invention.

FIG. 4. Characterization with different treatments for blocking unspecific binding sites in a polystyrene-based device according to the invention. Doxy-5 refers to the compound of formula (II) according to the invention.

FIG. 5. Immunoassay with a polystyrene-based device according to the invention on human CSF. ASm and ASf correspond to in vitro samples of monomeric and fibrillar AS, respectively, in PBS buffer. Samples F, B, C, G and E refer to CSF obtained from patients who do not register pathologies compatible with movement disorders.

FIG. 6. Optimization of conditions for Doxy-5 immobilization in BSA using glutaraldehyde, and discrimination results between Tau monomer (Tauₘ) and pre-formed fibrils (PFF).

FIG. 7. Results for comparison of Doxy-5 immobilization in BSA-glutaraldehyde with replacement of Doxy-5 with BSA on interaction with Tauₘ or PFF in different dilutions (p≤0.05). Black bars show the value for blank considered negative (OD<0.1).

FIG. 8. Comparative assay on the binding efficacies of Doxy-5 and commercial Doxycycline Hyclate (Doxy-Hy) to AS PFFs (p<0.0001).

### DETAILED DESCRIPTION OF THE INVENTION

The present invention refers to a device for detecting neurotoxic amyloid-type protein aggregates, based on the capability of doxycycline to bind to such protein aggregates.

As previously pointed out, AS aggregates (i.e., polymeric forms of AS) are considered the main biomarkers for PD, while Tau aggregates are considered promising biomarkers for AD. The device of the present invention thus has the advantageous property of being useful for detecting both PD and AD biomarkers in biological samples.

Correspondingly, the device of the invention comprises a doxycycline derivative immobilized on a surface.

Within the scope of the present disclosure, a doxycycline derivative is to be understood as a compound obtained by introducing minor modifications to doxycycline, while maintaining its capability of binding to neurotoxic amyloid-type protein aggregates such as AS and Tau aggregates. For example, such a doxycycline derivative can be a compound of formula (I):

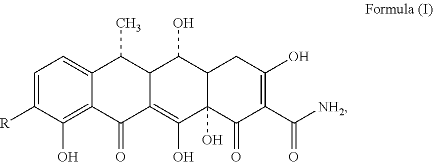

immobilized on a surface, wherein R is selected from the group consisting of:
i—a substituent selected from the group consisting of H, NH₂, COOH, (CH₂)ₙX, COOCH₂(CH₂)ₙX, wherein n=0-10 and X is selected from Cl, Br and I;
ii—an alkyl or aryl linker with a thiol end group;
iii—

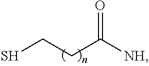

wherein n=0-10;
iv—

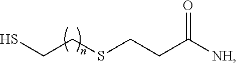

wherein n=0-10; and
v—

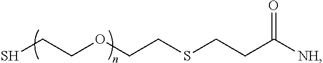

wherein n=0-10.

The alkyl or aryl linker with a thiol end group of the doxycycline derivative of formula (I) refers to any adequate alkyl chain or aryl group which includes a thiol group at its end.

In the case of the linker being an alkyl linker, the linker may be any alkyl hydrocarbon chain, either lineal or ramified, with a thiol group bound to the carbon atom which is the farthest from the bond of R to the aromatic ring of formula (I).

In the case of the linker being an aryl linker, the linker may be any aryl group, either with a thiol group bound directly to the aromatic system thereof, or comprising an alkyl substituent with a thiol group bound to the carbon atom which is the farthest from the bond of such an alkyl substituent to the aromatic system of the aryl group.

In a preferred embodiment, R is a substituent selected from the group consisting of H, NH₂, COOH, (CH₂)ₙX, COOCH₂(CH₂)ₙX, wherein n=0-10 and X is selected from Cl, Br and I.

In a particularly preferred embodiment, the doxycycline derivative is the compound of formula (II):

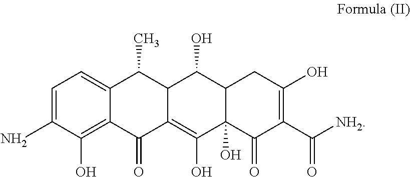 [SMILES: C[C@H]1c2ccc(N)c(O)c2C(=O)C2=C(O)[C@]3(O)C(=O)C(C(N)=O)=C(O)CC3[C@@H](O)C21]

The compound of formula (II) is also herein referred to as Doxy-5.

The device of the invention requires the doxycycline derivative to be immobilized on an adequate surface. By “immobilized”, a person of skill in the art will understand that the compound should be bound to the surface in a manner that will allow it to remain bound thereto even after several rinses. The manner in which the compound is immobilized on the surface will depend on the nature of the surface.

In an embodiment of the invention, the surface is a polymeric surface. When using this kind of surface, the doxycycline derivative is commonly immobilized thereon either by an adsorption process, i.e., the compound is bound to the surface by intermolecular forces, or by means of a blocking agent previously adsorbed on the polymeric surface, which the doxycycline derivative may be covalently bound to through an appropriate linker.

The term “blocking agent” should be understood as referring to a protein which readily binds to the polymeric surface of the device, such as those proteins used to block the surfaces of, for instance, ELISA plates. Such blocking agents are known to the person of skill in the art. However, the present inventors have surprisingly found that using bovine serum albumin (BSA) as blocking agent, the sensibility of the device for the detection of neurotoxic amyloid-type protein aggregates is greatly improved. Therefore, when the polymeric surface comprises a blocking agent adsorbed thereon, said blocking agent adsorbed onto the polymeric surface of the device is preferably BSA.

The linker through which the doxycycline derivative covalently binds to the blocking agent is a bi-functional small molecule comprising two reactive moieties in opposing ends of a hydrocarbon chain, so that one reactive moiety may covalently bind to the blocking agent adsorbed on the polymeric surface, and the other reactive moiety may bind covalently to the doxycycline derivative, thus immobilizing the doxycycline derivative on the polymeric surface. The selected linker will depend on the selected binding agent and doxycycline derivative, as a person of skill in the art will appreciate.

When the polymeric surface comprises a blocking agent adsorbed thereon, the doxycycline derivative is a doxycycline derivative which has been chemically modified to comprise a functional group capable of binding to the linker bound to the blocking agent adsorbed on the polymeric surface. For example, the doxycycline derivative able to bind to the linker bound to the blocking agent has the formula (I) as defined above.

In a preferred embodiment, the device comprises a polymeric surface onto which bovine serum albumin has been adsorbed, and wherein the doxycycline derivative of formula (II) is covalently bound to said bovine serum albumin through glutaraldehyde as linker.

In another preferred embodiment, no blocking agent is used, and the device comprises a polymeric surface onto which the doxycycline derivative has been immobilized by direct adsorption thereon, without the need of using a blocking agent.

The term “polymeric surface” is to be understood broadly, as referring to any surface comprising a polymer which the doxycycline derivative or the blocking agent may readily adsorb to. For example, the polymeric surface may comprise a polymer selected from the group consisting of polystyrene, a polystyrene/divinylbenzene copolymer, and other synthetic or natural polymers where doxycycline and derivatives thereof may be immobilized. Preferably, the polymeric surface comprises polystyrene.

In another embodiment of the invention, the surface is a functionalized metallic surface. The term “functionalized metallic surface” should be understood as referring to a metallic surface to which a functionalizing agent has been bound. The function of the functionalizing agent is to act as a linker between the doxycycline derivative, by binding covalently to both the metallic surface and the doxycycline derivative. Preferably, the functionalized metallic surface is a functionalized gold surface. More preferably, the functionalized metallic surface is a functionalized gold surface to which a mercapto acid is bound as a functionalizing agent, thus forming a self-assembled monolayer (SAM). Therefore, in this preferred embodiment, the functionalized gold surface comprises a SAM of a linear mercapto acid. In such an arrangement, the linear mercapto acid molecules bind to the gold surface by means of the terminal thiol moiety thereof in an ordered fashion, exposing the carboxylic acid moiety to which the doxycycline derivative may be covalently bound. Most preferably, the functionalized gold surface comprises a SAM of 3-mercaptopropionic acid.

When the surface is such a functionalized metallic surface, the doxycycline derivative is a doxycycline derivative which has been chemically modified to comprise a functional group capable of binding to the functionalizing agent of the functionalized metallic surface. For example, the doxycycline derivative able to bind to the functionalizing agent has the formula (I) as defined above.

In a particularly preferred embodiment, the device comprises the doxycycline derivative of formula (II) immobilized on a functionalized metallic surface, wherein the functionalized metallic surface is a functionalized gold surface, wherein the functionalized gold surface comprises a SAM of 3-mercaptopropionic acid, and wherein the doxycycline derivative of formula (II) is covalently bound to the SAM of 3-mercaptopropionic acid.

The device according to the invention can be prepared by techniques readily available for any person of skill in the art for immobilizing compounds on an adequate surface. It is thus another aspect of the invention to provide a method for preparing a device for detecting neurotoxic amyloid-type protein aggregates according to the invention, comprising:
i—providing a surface capable of immobilizing a doxycycline derivative; and
ii—contacting the surface capable of immobilizing a doxycycline derivative with a solution comprising a doxycycline derivative under conditions such that the doxycycline derivative is immobilized on the surface.

Providing a surface capable of immobilizing a doxycycline derivative comprises modifying the corresponding surface to make it capable of such an immobilization, if necessary.

In a particular embodiment, the surface is a polymeric surface. In such an embodiment, the surface is able to immobilize the doxycycline derivative by means of an adsorption process, without any further modifications.

However, in an alternative embodiment of this aspect of the invention, the polymeric surface is previously treated with a blocking agent which adsorbs thereon. Therefore, in said embodiment, step i of the method comprises the following sub-steps:
a) contacting the polymeric surface with a solution comprising a blocking agent so as to cause the adsorption of said blocking agent onto the polymeric surface, thus obtaining a blocked polymeric surface; and
b) contacting the blocked polymeric surface with a solution comprising a linker to obtain a polymeric surface capable of immobilizing a doxycycline derivative.

The polymeric surface capable of immobilizing a doxycycline derivative thus provided comprises the blocking agent adsorbed on the polymeric surface, with the linker covalently bound thereto offering a moiety to which the doxycycline derivative may readily covalently bind to. It is within the knowledge of a person of skill in the art to optimize the conditions upon which the adsorption of the blocking agent and the reaction with the linker take place.

The blocking agent and the linker share the same features as recited above in this description. In a preferred embodiment, the blocking agent is BSA, and the linker is glutaraldehyde.

In another particular embodiment, the surface is a functionalized metallic surface. In such an embodiment, the metallic surface must be functionalized so as to be able to immobilize a doxycycline derivative. Therefore, in such an embodiment, the method further comprises a step prior to step i, wherein a metallic surface is contacted with a solution comprising a functionalizing agent to obtain said functionalized metallic surface. Preferably, the metallic surface is a gold surface. Also preferably, the functionalizing agent is a mercapto acid. More preferably, the functionalizing agent is 3-mercaptopropionic acid. It is within the knowledge of a person of skill in the art to optimize the conditions upon which the functionalization of the metallic surface takes place.

The step of contacting the surface able for immobilizing a doxycycline derivative with a solution comprising a doxycycline derivative in conditions such that the doxycycline derivative is immobilized on the surface also depends on the selected surface.

In the embodiment wherein the surface is a polymeric surface, the step comprises submersing the corresponding surface in a solution comprising the doxycycline derivative in an appropriate concentration during an adequate amount of time for either the adsorption process or the reaction with the linker to be complete. In the particular embodiment wherein the polymeric surface was previously treated with the binding agent and the linker, the doxycycline derivative should also be a doxycycline derivative capable of binding to the linker bound to the blocking agent, preferably, by being a doxycycline derivative of formula (I) or, most preferably, by being a doxycycline derivative of formula (II).

In the embodiment wherein the surface is a functionalized metallic surface, the solution comprising the doxycycline derivative should also comprise any additional reagent necessary to form a covalent bond between the doxycycline derivative and the functionalized metallic surface. The doxycycline derivative should also be a doxycycline derivative capable of binding to the functionalizing agent of the functionalized metallic surface, preferably, by being a doxycycline derivative of formula (I) or, most preferably, by being a doxycycline derivative of formula (II). In the embodiment wherein the metallic surface is a gold surface and the functionalizing agent is 3-mercaptopropionic acid, the solution comprising the doxycycline derivative should comprise the necessary reagents for the doxycycline derivative to effectively bind to the carboxylic acid moiety of the 3-mercaptopropionic acid. For example, in such an embodiment the solution could also comprise 1-ethyl-3-(3-dimethylaminopropyl) carbodiimide hydrochloride (EDC) and N-hydroxysuccinimide (NHS).

Optimizing the conditions for the adsorption process or the reaction necessary for the doxycycline derivative to be immobilized on the selected surface is well within the skill of a person of skill in the art.

The device of the invention is intended for detecting neurotoxic amyloid-type protein aggregates in a sample. The term “neurotoxic amyloid-type protein aggregates” as used herein refers to protein aggregates associated with neurodegenerative diseases. Such aggregates are those typically observed accumulating in neurons and other sites of the nervous system during the progression of certain neurodegenerative diseases, and may thus be used as biomarkers for diagnosing such diseases. For example, the device may be used for detecting AS aggregates or Tau aggregates, associated with synucleinopathies such as PD and tauopathies such as AD, respectively, in such a sample. The signal emitted by the device on detection of said aggregates is related to the concentration thereof in said sample. Therefore, it is yet another aspect of this invention to provide an in vitro method for detecting and/or quantifying neurotoxic amyloid-type protein aggregates, comprising:
i—providing a device for detecting and/or quantifying neurotoxic amyloid-type protein aggregates according to the invention;
ii—contacting the device with a sample in which the presence of said aggregates is to be determined; and
iii—determining the presence and/or concentration of said aggregates by a detection technique.

The method takes advantage of the capability of the doxycycline derivatives of binding to said aggregates. By contacting a device comprising a doxycycline derivative immobilized on a surface with a sample comprising neurotoxic amyloid-type protein aggregates, said aggregates bind to the doxycycline derivative, which allows for their detection by an appropriate detection technique.

The detection technique used for detecting and/or quantifying the neurotoxic amyloid-type protein aggregates in the related method of the present invention may depend on the surface used for immobilizing the doxycycline derivative. For instance, the detection technique may be selected from the group consisting of an immunochemical assay and an electrochemical assay. In some embodiments, the detection technique comprises a sandwich immunoassay.

In a particular embodiment, the detection technique is an immunochemical assay, which requires the surface to be a polymeric surface, preferably, a polystyrene surface. Such immunochemical assay comprises contacting the device with the neurotoxic amyloid-type protein aggregates from the sample are bound to the immobilized doxycycline derivative, as obtained in step ii—of the method, with an antibody with specific affinity for the protein present in the bound aggregates, which may then be detected by a detection apparatus, either by itself or by means of yet another antibody.

For example, if the neurotoxic amyloid-type protein aggregates present in the sample are AS aggregates, their binding to the immobilized doxycycline can be detected by means of an anti-AS antibody. The binding of the anti-AS antibody to the bound AS can then be detected using an HRP-linked secondary antibody. Similarly, if the neurotoxic amyloid-type protein aggregates present in the sample are Tau aggregates, their binding to the immobilized doxycycline can be detected by means of an anti-Tau antibody. The binding of the anti-Tau antibody to the bound Tau can then be detected an HRP-linked secondary antibody. Such immunochemical assays are well within the skill of those in the art. However, the provision of a versatile method such as the one described herein, wherein the method that it can be applied to the detection and/or quantification of a series of neurotoxic amyloid-type protein aggregates associated to neurodegenerative, simply by varying the antibody used to detect the particular protein involved in the formation of the bound aggregates, clearly cannot be derived from the state of the art in an evident manner.

A person of skill in the art will appreciate that the detection technique described above is applicable when the surface of the device is a polymeric surface, regardless of whether the doxycycline derivative is immobilized on said polymeric surface by direct adsorption or through a blocking agent-linker bond, as described above.

In another particular embodiment, the detection technique is an electrochemical assay, which requires the surface to be a functionalized metallic surface, preferably, a functionalized gold surface. In such an embodiment, the metallic surface functions as an electrode in an apparatus for performing electrochemical measurements, such as a potentiostat. Preferably, the electrochemical assay uses a technique selected from the group consisting of CV and EIS for detecting and/or quantifying the neurotoxic amyloid-type protein aggregates. More preferably, the electrochemical assay uses CV for detecting and/or quantifying the neurotoxic amyloid-type protein aggregates.

In this embodiment of the invention, the specificity for each protein ((AS) aggregates and (Tau) aggregates) can then be assessed by the use of specific antibodies, the binding of which can also be assessed electrochemically. That is, an antibody with selective affinity for a specific protein (either AS or Tau) may be bonded to the corresponding aggregate bound to the doxycycline derivative, thus modifying the detected electrochemical signal. However, a positive signal in this sensor already indicates the presence of some of the aggregates, so it would be a determination at the patient point-of-care (POC), which already gives a positive diagnosis.

As mentioned above, the formation of neurotoxic amyloid-type protein aggregates which may be detected and/or quantified by means of the method of this aspect of the invention are observed several neurodegenerative diseases. In an embodiment, the method is for the detection and/or quantification of AS aggregates, which are associated with synucleinopathies such as PD, dementia with Lewy bodies (DLB) and multiple system atrophy (MSA). In another embodiment, the method is for the detection and/or quantification of Tau aggregates, which are associated with tauopathies such as AD, progressive supranuclear palsy (PSP), corticobasal degeneration (CBD), chronic traumatic encephalopathy (CTE) and Pick's disease (PiD).

Correspondingly, the method of the present invention can be used for early diagnosis of such neurodegenerative diseases. For such a utility, the sample in which the presence of the neurotoxic amyloid-type protein aggregates is to be determined is a sample from a subject for which the incidence of one of said diseases is suspected.

As used herein, the term diagnosis means detecting a disease or disorder or determining the stage or degree of a disease or disorder. The diagnostic methods may be used independently, or in combination with other diagnosing and/or staging methods known in the art for a particular disease or disorder. Additional diagnostic markers may be combined with the markers described herein for predicting the presence or absence or stage of a disease. For example, clinical factors of relevance to the diagnosis of a neurodegenerative disease include, but are not limited to, the patient's medical history, a physical examination, and other biomarkers.

As used herein, the term biological sample and sample can be used interchangeable and refers to a sample that has been obtained from a patient or subject. In some instances, the biological sample can be a tissue, cell(s), or a bodily fluid (e.g., blood, serum, synovial fluid, sputum, lung fluid, mucus, tears, lymphatic fluid, synovial fluid, cerebrospinal fluid (CSF), stool, saliva, amniotic fluid, amniotic cord blood, urine, vaginal fluid and semen). Preferably, such a sample is a cerebrospinal fluid (CSF) sample.

As used throughout, by subject is meant an individual, for example, an adult subject. Preferably, the subject is an animal, for example, a mammal such as a primate, and, more preferably, a human. Non-human primates are subjects as well. The term subject includes cats, dogs, reptiles, amphibians, livestock (for example, cattle, horses, pigs, sheep, goats, etc.) and laboratory animals (for example, ferret, chinchilla, mouse, rabbit, rat, gerbil, guinea pig, etc.). Thus, veterinary uses are contemplated herein.

It is yet another aspect of the invention to provide a kit for detecting and/or quantifying neurotoxic amyloid-type protein aggregates, comprising
i—a polymeric surface functionalized or ready to be functionalized with a doxycycline derivative; and
ii—detection reagents.

The term “polymeric surface functionalized or ready to be functionalized with a doxycycline derivative” is to be understood as referring to a polymeric surface as described above for the device of the invention, which either comprises the doxycycline derivative already immobilized thereon or is able to readily immobilize the doxycycline derivative upon contact with a solution comprising it.

The detection reagents comprise reagents known to a person of skill in the art to perform an immunochemical detection and/or quantification of the neurotoxic amyloid-type protein aggregates according to the in vitro method described above. Such detection reagents may comprise, for instance, secondary antibodies, stain reagents, etc. In a particular embodiment, the polymeric surface of the kit is ready to be functionalized with a doxycycline derivative, and the detection reagents comprise the doxycycline derivative as well.

In a preferred embodiment, the kit according to this aspect of the invention comprises a doxycycline derivative of formula (I), either immobilized on the polymeric surface or comprised within the detection reagents. More preferably, the doxycycline derivative is the doxycycline derivative of formula (II).

The kit according to this aspect of the invention could be operated by a laboratory technician and in medium complexity laboratories.

It is yet another aspect of the invention to provide a point-of-care (POC) biosensor for detecting biomarkers associated with PD, comprising an electrode which: (i) comprises a doxycycline derivative immobilized thereon, or (ii) is capable of immobilizing a doxycycline derivative thereon.

The term “an electrode which: (i) comprises a doxycycline derivative immobilized thereon, or (ii) is capable of immobilizing a doxycycline derivative thereon” is to be understood as referring to a functionalized metallic surface as described above for the device of the invention, which either comprises the doxycycline derivative already immobilized thereon or is able to readily immobilize the doxycycline derivative upon contact with a solution comprising it. Preferably, the POC biosensor comprises an electrode which comprises a doxycycline derivative immobilized thereon, more preferably, a doxycycline derivative of formula (I), most preferably, the doxycycline derivative of formula (II).

The present invention has the main advantage of being highly selective for detecting neurotoxic aggregated forms of proteins such as AS and Tau over monomeric forms thereof, while being easy to implement, and not requiring particularly expensive or sophisticated equipment. Therefore, it shows great potential use as a POC device for early detection of biomarkers associated with diseases such as PD and AD, which could lead to an early diagnosis of said diseases, thus providing a much better prognosis thereof.

### EXAMPLES

The present invention is further illustrated by the following Examples, which are not intended to limit the scope thereof. Instead, the Examples set forth below should be understood only as exemplary embodiments for better taking into practice the present invention.

### Example 1—Organic Synthesis of the Compound of Formula (II)

### General Experimental Procedures

NMR spectra were recorded at 500 MHZ (¹H) or 125.7 MHz (¹³C) or at 300 MHZ (¹H) or 75.6 MHz (¹³C). Chemical shifts (δ, in ppm) were referred to an internal standard (Me₄Si in CDCl₃ (δ0.0) for 1H and CDCl₃ (δ: 77.0) for ¹³C) or to a residual solvent peak. Data multiplicities are indicated as s (singlet), d (doublet), t (triplet), q (quartet), m (multiplet), br (broad); coupling constants (J) given in Hertz (Hz). Assignments of ¹H and ¹³C NMR spectra were assisted by 2D ¹H-COSY or NOESY, and 2D ¹H-¹³C HSQC. High-resolution mass spectra (HRMS) were obtained using the electrospray ionization (ESI) technique and Q-TOF detection. Analytical thin-layer chromatography (TLC) was carried out on silica gel 60 F254 aluminum-supported plates (layer thickness 0.2 mm) and on silica gel 60 RP F254S aluminum-supported plates. The spots were visualized by exposure to UV light and by charring with Ce/Mo stain. Column chromatography was carried out with silica gel 60 (230-400 mesh) or, for reverse phase, octadecyl-functionalized silica gel was employed as stationary phase. The chromatography solvents or the stepwise solvent polarity gradients used are specified for each individual compound. Optical rotations were measured at the sodium D line at room temperature in a 1 dm cell, in the solvent indicated. Unless otherwise noted, all commercially available compounds were used as obtained from suppliers without further purification.

Doxycycline Free Form (D-1) from Doxycycline Hydrochloride

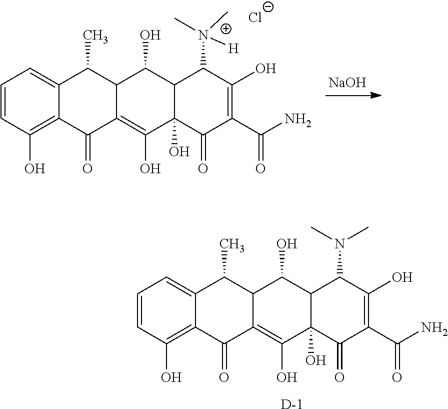

Doxycycline hydrochloride (2.0 g, [α]_{D}²⁰=−113.5 (c 1, 10 mM HCL in MeOH) was dissolved in distilled water (6 mL) and 1 M aqueous NaOH was added dropwise up to PH≈5. At this point a white solid was formed. The solid was filtered and dissolved in methanol (20 mL). The solution was stirred for 20 min until a new white precipitate appeared. The solid was filtered and dried, affording doxycycline free form (D−1, 1.3 g, 75%); [α]_{D}²⁰=+250.7 (c 1, THF), ¹H NMR ((CD₃)₂CO, 500 MHZ) δ: 7.53 (t, 1H, J_{7,8}=J_{8,9}=8.1 Hz, H-8), 7.02 (d, 1H, J_{8,9}=8.1 Hz, H-9), 6.82 (d, 1H, J_{7,8}=8.1 Hz, H-7), 4.21 (brt, 1H, J_{5a,5}=J_{4,5}≈4.0 Hz, H-5), 3.59 (brd, 1H, J_{4a,4}=9.6 Hz, H-4), 2.93 (dq, 1H, J_{6,Me}=6.7, J_{5a,6}=12.8 Hz, H-6), 2.74 (dd, 1H, J_{5a,5}=4.0, J_{5a,6}=12.8 Hz, H-5a), 2.63 (dd, 1H, J_{4a,4}=9.6, J_{4a,5}=4.0 Hz, H-4a), 2.53 (s, 6H, N(CH₃)₂), 1.61 (d, 3H, J_{6,Me}=6.7 Hz, CH₃); ¹³C NMR ((CD₃)₂CO, 125.7 MHZ) δ: 194.7 (C-1,3, 11), 174.9, 174.5 (C-12, CONH₂), 163.4 (C-10), 148.7 (C-6a), 137.6 (C-8), 117.4, 116.8, 116.4 (C-7,9,10a), 106.4 (C-11a), 92.0 (C-2), 76.0 (C-12a), 69.9 (C-5), 66.2 (C-4), 48.9 (C-4a), 48.2 (C-5a), 42.6 (N(CH₃)₂), 38.5 (C-6), 16.7 (CH₃).

This same procedure was applied to doxycycline hyclate yielding 69% of pure D-1.

### Synthesis of Doxycycline Methyl Iodide Salt (D−2)

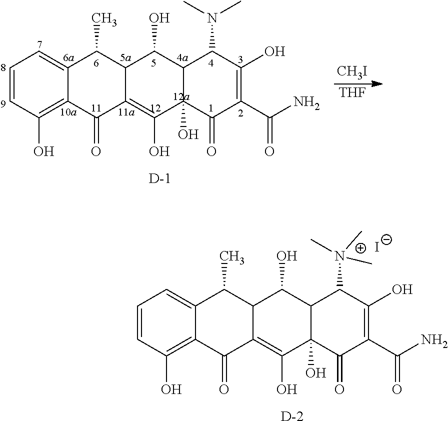

To a solution of D-1 (2.0 g, 4.5 mmol) in dry THF (40 mL) was added dropwise methyl iodide (2.5 mL, 40 mmol) at room temperature and under Ar atmosphere. The reaction was stirred at 45° C. for 24 h, and the solvent was removed by evaporation under reduced pressure. The resulting solid was washed with anhydrous CH₂Cl₂ (15 mL) and dried to give D-2 (2.6 g, 98%). [α]_{D}²⁰+31.2 (c 1.0, THF); ¹H NMR ((CD₃)₂CO, 200 MHZ) δ: 7.55 (t, 1H, J_{7,8}=J_{8,9}=8.0 Hz, H-8), 6.97 (d, 1H, J_{8,9}≈8.0 Hz, H-7), 6.86 (d, 1H, J_{7,8}≈8.1 Hz, H-9), 5.44 (s, 1H, OH), 3.89 (brt, 1H, H-5), 3.69 (s, 9H, N(CH₃)₃), 3.53 (brd, 1H, H-4), 2.97-2.60 (m, 3H, H-4a,5a,6), 1.56 (d, 3H, J_{6,Me}=6.4 Hz, CH₃); HRMS (ESI) m/z [M]⁺ calcd for C₂₃H₂₇N₂O₅ 459.1762, found 459.1762.

### Synthesis of 4-dedimethylaminodoxycycline D-3

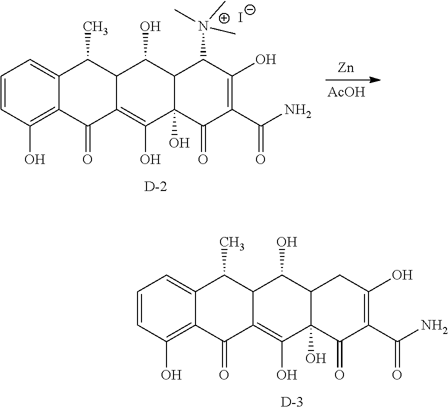

To a solution of D-2 (1 g, 1.7 mmol) in 50% (v/v) aqueous acetic acid (30 ml) was added zinc dust (0.6 g, 9.2 mmol) and the mixture was stirred at room temperature for 20 min. The suspension was filtered through a pad of celite. The filtrate was diluted with water (100 mL) containing concentrated HCl (1 mL) and this mixture was stirred in an ice bath for 1 h. The solid formed was filtered and dried in vacuum. This amorphous solid was characterized as D-3 (0.48 g, 70%); [α]_{D}²⁰−50.8 (c 1.0, acetone); ¹H NMR ((CD₃)₂CO, 500 MHz) δ: 7.51 (t, 1H, J_{7,8}=J_{8,9}=8.0 Hz, H-8), 6.96 (d, 1H, J_{8,9}=8.0 Hz, H-9), 6.84 (d, 1H, J_{7,8}=8.0 Hz, H-7), 4.41 (d, 1H, J_{5,OH}=8.5 Hz, OH), 3.79 (br q, 1H, J_{4a,5}=9.5, J_{5a,5}=7.7, J_{5,OH}=8.5 Hz, H-5), 3.06 (dd, 1H, J_{4a,4}=5.5, J_{4,4′}=18.6 Hz, H-4), 2.97 (dd, 1H, J_{4a,4′}=2.9, J_{4,4′}=18.6 Hz, H-4′), 2.79 (m, 1H, J_{6,Me}=6.8, J_{5a,6}=12.5 Hz, H-6), 2.51 (dd, 1H, J_{5a,5}=7.7, J_{5a,6}=12.5 Hz, H-5a), 2.47 (ddd, 1H, J_{4,4}=5.5, J_{4a,4}′=2.9, J_{4,5}=9.5 Hz, H-4a), 1.57 (d, 3H, J_{6,Me}=6.8 Hz, CH₃); ¹³C NMR (CD₃)₂CO, 125.7 MHZ) δ: 195.9, 194.6, 193.2 (C-1,3,11), 176.0, 174.9 (C-12, CONH₂), 163.1 (C-10), 149.1 (C-6a), 137.4 (C-8), 116.8, 116.7, 116.6 (C-7,9,10a), 107.4 (C-11a), 99.7 (C-2), 75.7 (C-12a), 69.6 (C-5), 44.4 (C-4a), 47.6 (C-5a), 39.5 (C-6), 30.6 (C-4), 16.4 (CH₃); HRMS (ESI) m/z [M+Na]⁺ calcd for C₂₀H₁₉NNaO₈ 424.0998, found 424.1003.

### Synthesis of 4-dedimethylamino-9-nitro doxycycline D-4

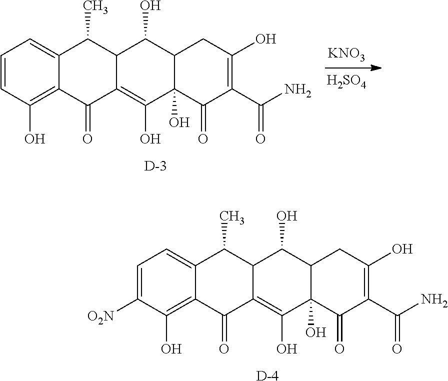

To D-3 (0.5 g, 1.2 mmol) was slowly added 97% H₂SO₄ (4 mL), which had been previously cooled in an ice bath. To this solution KNO₃ (0.16 g, 1.6 mmol) was added and the mixture was stirred at 0° C. for 2 h. The reaction was diluted with cold methanol (5 mL) and upon addition of water (35 mL) a precipitate was formed. The brownish solid was filtered, dried in vacuum and then dissolved in acetone (4 mL). Upon addition of dichloromethane (15 mL) a black precipitate appeared. This mixture was treated with activated carbon with stirring for 20 min, and then filtered through a celite pad. The solid was discarded and the filtrate was slowly diluted with hexane (70 mL) to induced the precipitation of D-4 (0.35 g, 65%) as a yellow solid; [α]_{D}²⁰−6.5 (c 1.0, acetone); ¹H NMR (CD₃)₂CO, 200 MHZ) δ: 8.15 (d, 1H, J_{7,8}=8.6 Hz, H-8), 7.17 (d, 1H, J_{7,8}=8.6 Hz, H-7), 3.83 (dd, 1H, J_{4,5}=11.4, J_{5a,5}=7.8 Hz, H-5), 3.00-2.91 (m, 3H, H-4, H-4′ and H-6), 2.61 (dd, 1H, J_{5a,6}=12.5, J_{5a,5}=7.8 Hz, H-5a), 2.47 (ddd, 1H, J_{4a,4}=5.3, J_{4a,4′}=3.3, J_{4,5}=11.4 Hz, H-4a), 1.60 (d, 3H, J_{6,Me}=6.8 Hz, CH₃); ¹³C NMR (CD₃)₂CO, 50.3 MHZ) δ: 165.5 (C-12, CONH₂), 155.1 (C-7), 137.4 (C-6a), 132.1 (C-9), 118.5, 118.2 (C-9,10a) 116.5 (C-8), 107.5 (C-11a), 94.6 (C-2), 75.6 (C-12a), 69.2 (C-5), 46.6 (C-5a), 44.2 (C-4a), 38.8 (C-6), 33.4 (C-4), 16.2 (CH₃); HRMS (ESI) m/z [M+Na]⁺ calcd for C₂₀H₁₈N₂NaO₁₀ 469.0854, found 469.0854.

### Synthesis of 9-amino-4-dedimethylamino doxycycline Doxy-5 (Compound of Formula (II))

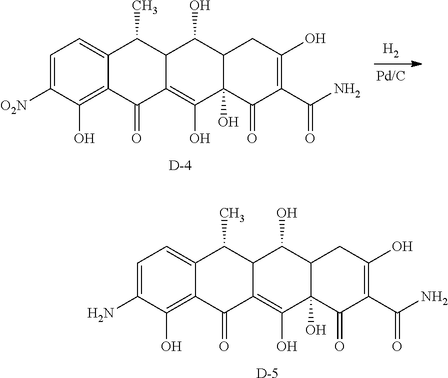

To a solution of D-4 (0.2 g, 0.5 mmol) in methanol (6 mL) containing 0.1% concentrated HCl was added 10% Pd/C (30 mg) and the mixture was treated with hydrogen at 44 psi and room temperature for 20 h. The mixture was filtered through a celite pad and the residue washed with methanol. The filtrate and washing liquors were pooled and concentrated. The resulting residue was dissolved in ethanol (2 mL) and precipitation was induced upon dropwise addition of ethyl acetate (30 mL), to afford Doxy-5 (125 mg, 60%) as a slightly grey solid; [α]_{D}²⁰−30.0 (c 0.5, MeOH); ¹H NMR (CD₃OD, 500 MHZ) δ: 7.61 (d, 1H, J_{7,8}=8.3 Hz, H-8), 7.10 (d, 1H, J_{7,8}=8.3 Hz, H-7), 3.67 (dd, 1H, J_{4a,5}=10.8, J_{5a,5}=8.0 Hz, H-5), 3.05 (dd, 1H, J_{4a,4}=5.5, J_{4,4′}=18.6 Hz, H-4), 2.92 (dd, 1H, J_{4a,4′}=2.4, J_{4,4′}=18.6 Hz, H-4′), 2.78 (m, 1H, J_{6,Me}=6.9, J_{5a,6}=12.4 Hz, H-6), 2.44 (dd, 1H, J_{5a,5}=8.0, J_{5a,6}=12.4 Hz, H-5a), 2.32 (ddd, 1H, J_{4a,4}=5.5, J_{4a,4′}=2.4, J_{4a,5}=10.8 Hz, H-4a), 1.55 (d, 3H, J_{6,Me}=6.9 Hz, CH₃); ¹³C NMR (CD₃OD, 125.7 MHZ) δ: 196.5, 194.5×2 (C-1,3,11), 177.2, 175.04 (CONH₂, C-12), 155.8 (C-10), 150.4 (C-6a), 130.9 (C-8), 118.9, 118.1 (C-9,10a) 117.3 (C-7), 108.0 (C-11a), 99.5 (C-2), 76.0 (C-12a), 69.8 (C-5), 47.7 (C-5a), 44.9 (C-4a), 40.0 (C-6), 31.3 (C-4), 16.2 (CH₃); HRMS (ESI) m/z [M]⁺ calcd for C₂₀H₂₁N₂O₈ 417.1292; found 417.1295.

### Example 2—Production of AS Aggregates as Biomarker Analyte

### 2.1. Expression and Purification of Human AS

Human AS was expressed in E. coli (BL21) using the pT7-7 plasmid which contains the codifying gene for the protein. The purification was performed by means of a selective precipitation with (NH₄)₂SO₄ and anionic exchange chromatography. The purity of the protein was confirmed by electrophoresis under denaturalizing conditions (SDS-PAGE). The AS solution was prepared in a HEPES 20 mM, 150 mM NaCl buffer, at pH 7.4. The sample was filtered and centrifuged during 30 minutes at 12000×g to remove any preformed aggregates. The concentration of AS was determined by spectrophotometry using its molar absorption coefficient.

### 2.2. Preparation of Aggregated AS Species

Aggregated AS species were obtained by incubating an AS solution (70 μM) in a controlled-temperature orbital agitator (600 rpm, 37° C.) during 24 h, as previously reported (Ávila C. L., et al. (2014).

### 2.3. Determination of the Kinetics of Amyloid Aggregation

The kinetic determination was carried out by the Congo Red (CR) and Thioflavin T (ThT) techniques, which bind specifically to the amyloid fibers. Free CR exhibits an absorption maximum at 490 nm, whereas when it is bound to amyloid fibers the peak shifts to 520 nm, additionally to exhibiting an increase in the intensity of the absorption band. When ThT binds to amyloid fibers, it goes from having an excitation maximum at 415 nm to having a much more intense maximum at 450 nm with a fluorescence emission wavelength at 480 nm, based on the teachings of LeVine (1999), optimized as described in Ávila C. L., et al. (2014).

### Example 3—Immobilization of a Doxycycline Derivative (Doxy-5) on a Conductive Surface for Electrochemical Detection of AS Aggregates

A comparative study for different crosslinking agents for the immobilization of Doxy-5 on gold conductive surfaces was performed. 3-mercaptopropionic acid (MPA), which is able to form self-assembled monolayers (SAM) on the substrate thus facilitating its functionalization, was used. Gold was chosen because it is inert for the molecule of interest. Additionally, the use of other agents such as 1-ethyl-3-(3-dimethylaminopropyl) carbodiimide hydrochloride (EDC) and N-hydroxysuccinimide (NHS), either for generating or activating present moieties, is necessary for the formation of covalent bonds with Doxy-5. Concentrations and incubation times were optimized in each modification stage of the gold surface.

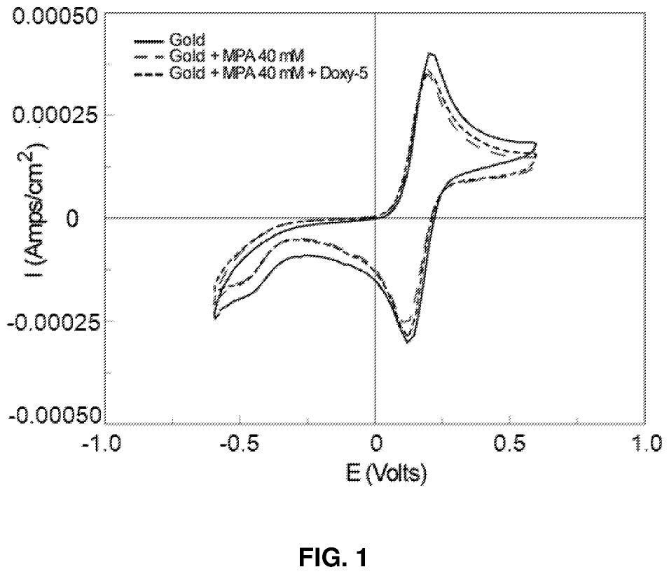

Cyclic voltammetry (CV) measurements were first carried out to characterize each of the immobilization stages on a gold substrate. Said measurements were carried out in a cell with tripolar configuration using a platinum electrode as counter electrode, a silver/silver chloride reference electrode, and the modified gold electrodes as working electrodes. The electrolyte [Fe(CN)₆]K₄, containing the Fe³⁺/Fe²⁺ redox pair, was used in every characterization assay. With this technique, the optimal polarization voltage value was determined for electrochemical impedance spectroscopy (EIS) measurements, that is, the voltage for which the biosensor exhibits its most sensitivity and specificity in the interaction between AS aggregated species and Doxy-5 immobilized on the conductive electrodes. CV and EIS measurements were also performed in each immobilization stage using phosphate buffer (PBS) at pH 7.4 (FIG. 1).

For the electrochemical measurements, a Solartron® 1287 potentiostat was used, as well as a Solarton® 1250 Frequency Response Analyzer. For data processing, the software provided by the manufacturer was used (CorrWare®, CorrView®, ZPlot® y ZView®). Measurements were carried out at room temperature. The biosensor was calibrated for increasing concentrations of toxic AS species.

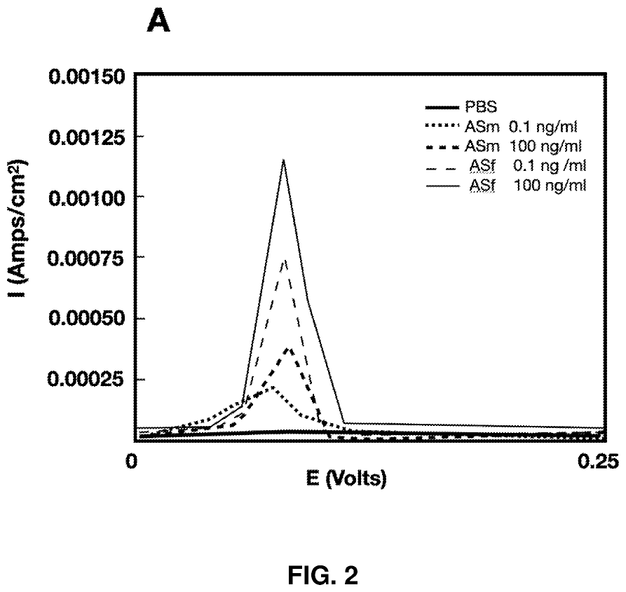

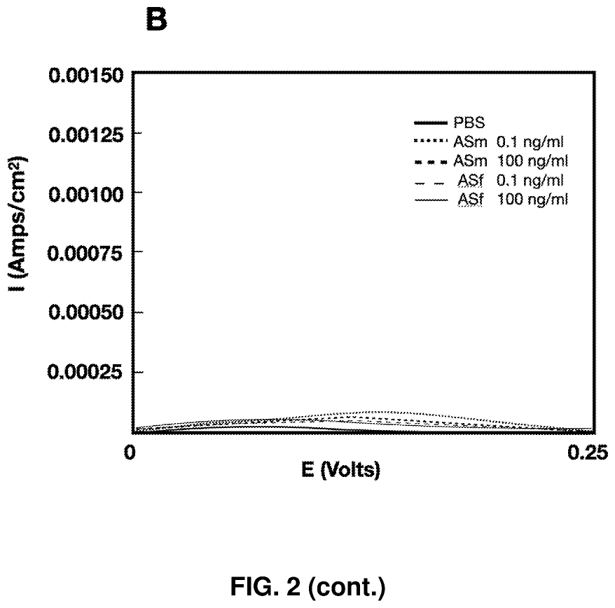

The electrochemical measurements of AS at different concentrations (0.1 and 100 ng/ml) were assayed with the gold biosensors modified with Doxy-5 prepared according to example 3.1. The impedance determinations were carried out in a range of frequencies from 0.1 to 65000 Hz using an alternating voltage of 10 mV overlapped at different potentials (0 V and −0.7 V). CV measurements were carried out in a potential range from −0.6 V to 0.6 V and a sweep rate of 30 mV/seg. The CV assays show, first, the non-existing interference of the monomer. The responses obtained for the monomer are much lower than the ones obtained for the AS fibers. In fact, at monomer concentrations of 100 ng/ml, the response is lower than the one obtained for the fibers at a concentration of 0.1 ng/mL. Second, a clear electrochemical response of the sensor in the presence of the AS fibers was observed. An increase in the oxidation peak of the voltammogram was observed from concentrations as low as 100 μg/mL to concentrations over 100 ng/ml (FIG. 2).

CV has proven to be a selective technique, since the presence of AS fibers is selectively recognized over the monomeric variant.

### Example 4—Immobilization of Doxy-5 on a Polymeric Surface by Direct Adsorption and Immunochemical Detection of AS Aggregates

The design is based on an immunoassay with a conventional sandwich system involving the capture of fibrillar species of AS by Doxy-5, followed by detection with two antibodies: an anti-human AS antibody, and an antibody conjugated to a peroxidase. Briefly, the compound of formula (II) is incubated in polystyrene microplates wherein it is adsorbed. After several rinses, to eliminate any non-adsorbed molecules, the system is incubated with a primary anti-human AS rabbit antibody. After rinsing, the system is then incubated with a secondary anti-rabbit antibody conjugated to peroxidase. The conjugated antibody reacts specifically with the primary antibodies which are bound to the immunocaptured AS. The unbound conjugated antibody is eliminated by rinsing, and the presence of peroxidase is revealed by the addition of tetramethylbenzidine (TMB) as a chromogenic substrate. After incubating, a blue color develops, whose intensity depends on the concentration and the affinity of Doxy-5 to the AS species. The enzymatic reaction is stopped by the addition of sulfuric acid, which produces a color shift towards yellow which is spectrometrically quantifiable at 450 nm.

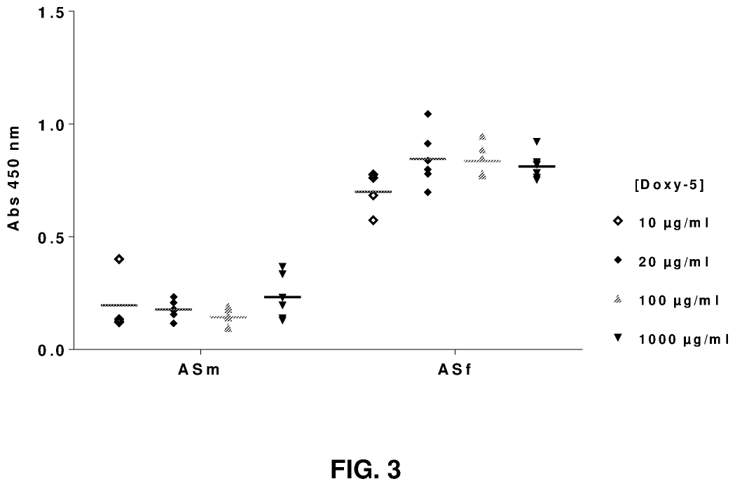

The effectiveness of this detection platform may be affected by different parameters, such as the concentration of the compound of formula (II) and the antibodies, the use of different proteins and their concentrations for blocking unspecific sites, as well as the temperatures and incubation times for each stage. To obtain the best analytical performance for this platform, assays were carried out to optimize said parameters (FIG. 3).

The specificity of the commercially available primary antibodies (Ab 138501, Ab209538, Ab27766, Abcam and Sc-7011-R, Santa Cruz) for the different AS species (monomeric, oligomeric, fibrillar) was validated. Every antibody showed picomolar affinity for every molecular AS form. The highest sensitivity was observed for the polyclonal antibody (Sc-7011-R, Santa Cruz) and for one of the monoclonal antibodies (Ab209538). The anti-AS antibodies did not show significant differences in the dosed values for fibrillar and monomeric AS. This is decisive in the design of this enzyme-immunoassay wherein the bioreceptor is the responsible of the specificity for fibrillar AS over monomeric AS. The concentrations of the primary and secondary antibodies were also optimized (Ab 32460, Invitrogen and Ab 6721, Abcam).

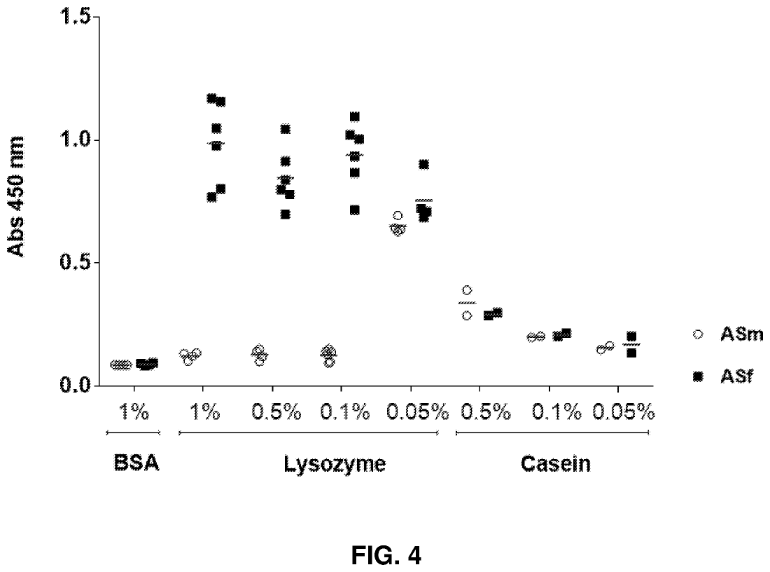

The immobilization of the compound of formula (II) was analyzed on Microlon® polystyrene plates (96 well, High binding, Greiner Bio-One). The concentration of the compound of formula (II) was characterized in combination with the use of different proteins and their concentrations for blocking unspecific sites. After assaying different blocking agents, such as bovine serum albumin (BSA), lysozyme and casein in concentrations ranging from 0.05% to 1%, it was established that lysozyme was the best option, with a concentration of 0.1% (FIG. 4).

The protocol was defined as follows:

The 96-well polystyrene plate was sensitized by incubation overnight a 4° C. and protected from light with 20 μg/mL of the compound of formula (II) in 20 mM sodium acetate pH 5 (50 μL/well). The plate was rinsed with PBST (PBS 10 mM pH 7.4+Tween-80 0.01%) and blocked with 150 μL/well of a 0.1% lysozyme solution in PBS 10 mM pH 7.4 during 1 h at 37° C. After rinsing, 50 μL of the samples with the analytes of interest prepared in 10 mM PBS pH 7.4 were added to each well, and the plate was incubated at 37° C. for 2 h. The plate is then rinsed again, and 50 μL of the anti-AS antibody diluted to 1 μg/ml in 10% bovine fetal serum (BFS in 10 mM PBS pH 7.4) were added to each well, after which the plate was incubated 1.5 h. The plate was rinsed once more and incubated at 37° C. during 1 h with 50 μL/well of the antibody conjugated with peroxidase diluted to 1 μg/ml in 10% bovine fetal serum. The plate is rinsed and incubated with 50 μL/well of the substrate (TMB) at room temperature for 15 minutes. The reaction is stopped with 25 μL of a 1 M H₂SO₄ solution. Immediately thereafter, the developed absorbance is measured with a TECAN Spark microplate reader.

The results were confirmed with at least 3 independent experiments, each by triplicate.

The focus of the immunoassay takes advantage of the generation of a colored compound by action of the peroxidase on the substrate (TMB), which is proportional to the concentration of the AS analytes captured by Doxy-5. The absorbance readings at 450 nm were acquired with a TECAN Spark microplate reader.

Standard solutions of monomeric and fibrillar AS, in a broad concentration range, were incubated in the wells which were previously sensitized with Doxy-5 as indicated in example 3.2 above. The system exhibited nanomolar affinity towards fibrillar AS, with no cross-reaction with native AS which was included as a negative control. Concentrations over 30 ng/mL showed the best differential reactivity between the aggregated and monomeric species of AS using the protocol described in example 3.2.

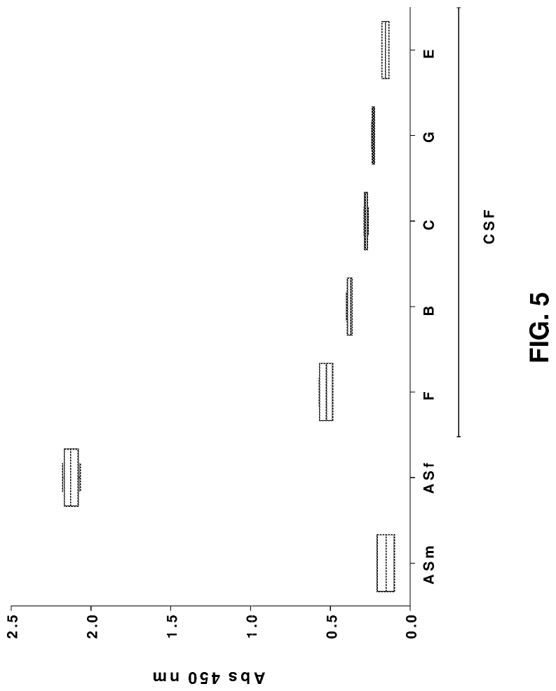

Additionally, the use of the platform was explored on samples of CSF of patients who do not register pathologies compatible with movement disorders. All of the samples were centrifuged at 2000 g for 10 minutes to eliminate the cells and debris, and were then stored in aliquots at −80° C. until they were analyzed. The assayed biological fluids exhibited values in the order of absorbance readings of the AS samples used as a negative control (FIG. 5).

### Example 5—Immobilization of Doxy-5 on a Polymeric Surface by Indirect Binding to BSA and Immunochemical Detection of Tau Aggregates

The sensitization of the wells was carried out as follows: first, the binding sites of the microplate were blocked with albumin. Afterwards, compound of formula (II) was covalently linked to albumin by using glutaraldehyde as the linker.

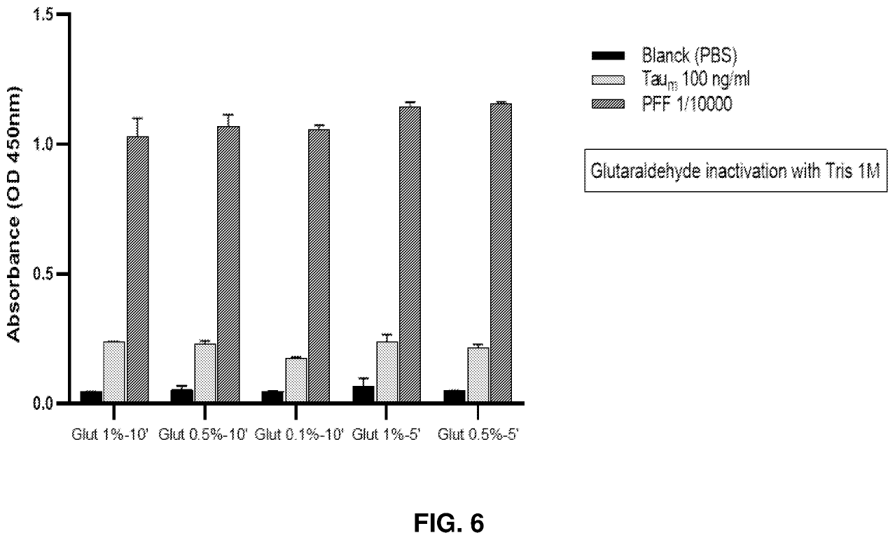

For this purpose, a 96-well polystyrene microplate was incubated with 150 μL/well of 1% bovine serum albumin (BSA) overnight at 4° C. in a humid chamber. Then, after three cycles of washing with PBST, 50 μL/well of glutaraldehyde at different concentrations (0.1%, 0.5% and 1%) were added to the wells and the plate was kept during specified times (either 10 minutes for every concentration, or 5 minutes for 0.5% and 1%) under a hood. Wells were then washed three times with PBS, and 50 μL/well of Doxy-5 (40 μg/ml) were added. The plate was then incubated at 37° C. for 30 minutes, protected from light. Afterwards, the wells were washed again with PBST, and 50 μL/well of 1M TRIS was added as a glutaraldehyde-inactivating agent. The microplate was then incubated at 37° C. for 30 minutes. After this incubation, wells were subsequently washed with PBST, and 50 μL/well of Tau samples (monomer (Tauₘ) and pre-formed fibrils (PFF)) were added, and the microplate was incubated at 37° C. for 2 hours. The concentration for Tauₘ was 100 ng/ml (5 ng/well) and PFF was proved in 1/10000 dilution. Then, wells were washed three times with PBST, and 50 μL/well of a 1/2000 solution of anti-Tau antibody (ab80579, abcam) diluted in PBS-FBS 10% was added. The microplate was then incubated at 37° C. for 1.5 hours. Following this, wells were washed with PBST five times, and 50 μL/well of HRP-linked secondary antibody (ab6789, abcam) (1/75000) was added. The microplate was then incubated at 37° C. for 1 hour, and afterwards the excess of secondary antibody was removed by washing with PBST five times. Lastly, 50 μL/well of TMB substrate was added and incubated for 10 minutes at room temperature, protected from light. To stop the reaction, 25 μL/well of 2N H₂SO₄ were added, and the microplate was immediately read on a microplate reader (Tecan Spark, Austria) at the wavelength of 450 nm. All the different conditions proved with glutaraldehyde showed similar results, and 0.1%-10′ was selected for further assays because it contains the lowest concentration. Doxy-5 was able to discriminate between Tauₘ and PFF in these conditions, showing a higher signal for PFF compared with Tauₘ and PBS (working buffer) (FIG. 6).

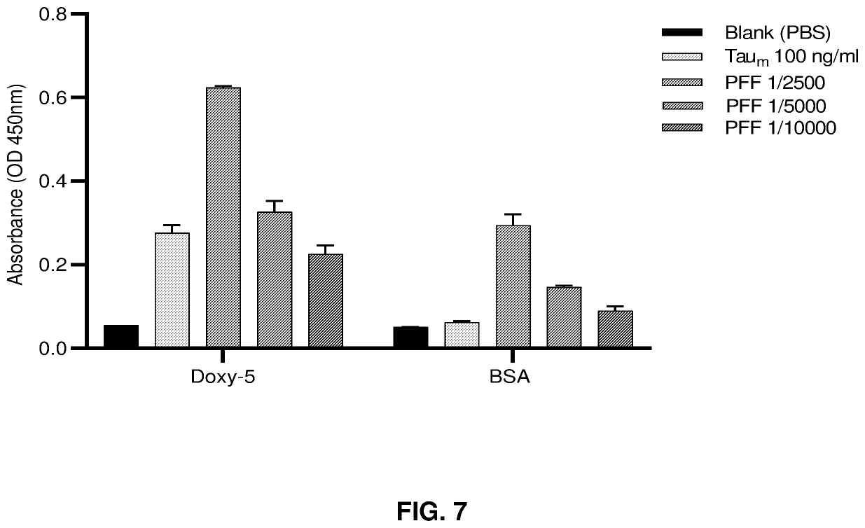

Additionally, whether PFF is binding to Doxy-5 or to remaining reactive sites from BSA treated with glutaraldehyde was evaluated. To this end, after adding glutaraldehyde in the protocol described above, Doxy-5 was replaced with BSA 0.1%. Then, samples of Tauₘ or PFF in different dilutions (1/2500, 1/5000, 1/10000) were added and the assay continued as described. Using BSA instead of Doxy-5, signal for PFF was significantly lower compared to the signal obtained with Doxy-5 (FIG. 7).

### Example 6—Comparative Binding Effectiveness of Doxycycline and Doxy-5 to Alpha-Synuclein Aggregates

Both doxycycline hyclate and Doxy-5 were immobilized on polystyrene microplates and contacted with AS aggregates (PPF) according to the protocol described in Example 4, using a concentration for the AS aggregates of 20 ng/ml. The binding of the AS aggregates to each molecule was determined as described in Example 4 as well.

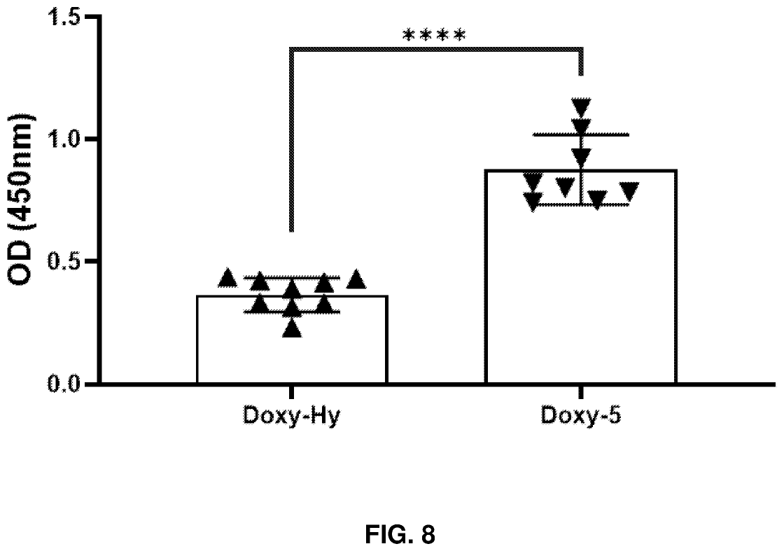

FIG. 8 shows that Doxy-5 is more effective than doxycycline hyclate at binding to amyloid aggregated species of AS. While a positive signal is observed with doxycycline hyclate, the signal obtained with Doxy-5 is significantly higher, suggesting that Doxy-5 binds with higher affinity to AS PFF than doxycycline hyclate. The results indicate that Doxy-5 is a superior capture element compared to conventional doxycycline hyclate, demonstrating higher selectivity for the target analyte. These findings underscore the importance of chemical modification of this tetracycline to develop a device with enhanced sensitivity. The removal of the bulky dimethylamine from position C4 of doxycycline resulted in an enhanced affinity of the molecule for amyloid aggregated species of AS.

### REFERENCES

Atik A, et al. Alpha-Synuclein as a Biomarker for Parkinson's Disease. Brain Pathol. 2016 May; 26 (3): 410-8. doi: 10.1111/bpa.12370.
Ávila C. L., et al. Structural Characterization of Heparin-induced Glyceraldehyde-3-phosphate Dehydrogenase Protofibrils Preventing α-Synuclein Oligomeric Species Toxicity, Journal of Biological Chemistry, Volume 289, Issue 20, 2014, Pages 13838-13850, ISSN 0021-9258, https://doi.org/10.1074/jbc.M113.544288.
Eusebi P, et al. Diagnostic utility of CSF α-synuclein species in Parkinson's disease: protocol for a systematic review and meta-analysis. BMJ Open 2016; 6:e011113. doi: 10.1136/bmjopen-2016-011113.
Ganguly U, et al. Alpha-Synuclein as a Biomarker of Parkinson's Disease: Good, but Not Good Enough. Frontiers in Aging Neuroscience. 2021; 13. DOI=10.3389/fnagi.2021.702639.
Golan H, et al. Nuclear imaging in Parkinson's disease: The past, the present, and the future. Journal of the Neurological Sciences. Volume 436. 2022. 120220.ISSN 0022-510X. https://doi.org/10.1016/j.jns.2022.120220
González-Lizárraga, F., et al. Repurposing doxycycline for synucleinopathies: remodelling of α-synuclein oligomers towards non-toxic parallel beta-sheet structured species. Sci Rep 7, 41755 (2017). https://doi.org/10.1038/srep41755.
Jang et al. α-Synuclein Oligomer Detection with Aptamer Switch on Reduced Graphene Oxide Electrode. Nanomaterials 2020, 10, 832; doi: 10.3390/nano10050832.
Kuzkina, A., et al. Diagnostic value of skin RT-QuIC in Parkinson's disease: a two-laboratory study. npj Parkinsons Dis. 7, 99 (2021). https://doi.org/10.1038/s41531-021-00242-2
Luan, M, et al. (2022), Diagnostic Value of Salivary Real-Time Quaking-Induced Conversion in Parkinson's Disease and Multiple System Atrophy. Mov Disord. https://doi.org/10.1002/mds.28976
Medina, L. et al. Doxycycline Interferes With Tau Aggregation and Reduces Its Neuronal Toxicity. Frontiers in aging neuroscience 13, 635760, doi: 10.3389/fnagi.2021.635760 (2021)
Moore, R. Y., et al. Extrastriatal monoamine neuron function in Parkinson's disease: an 18F-dopa PET study. Neurobiol Dis 29, 381-390 (2008).
Mounsey, A. L. & Zeitler, M. R. Cerebrospinal Fluid Biomarkers for Detection of Alzheimer Disease in Patients with Mild Cognitive Impairment. Am Fam Physician 97, 714-715 (2018).
Pavese, N., et al. Progression of monoaminergic dysfunction in Parkinson's disease: a longitudinal 18F-dopa PET study. Neuroimage 56, 1463-1468 (2011).
Poggiolini I, et al. Diagnostic value of cerebrospinal fluid alpha-synuclein seed quantification in synucleinopathies, Brain, Volume 145, Issue 2, February 2022, Pages 584-595, https://doi.org/10.1093/brain/awab431
Siderowf, A. et al. Assessment of heterogeneity among participants in the Parkinson's Progression Markers Initiative cohort using α-synuclein seed amplification: a cross-sectional study. The Lancet Neurology, Volume 22, Issue 5 (2023), Pages 407-417, https://doi.org/10.1016/S1474-4422 (23) 00109-6.
Yang, S Y., et al. Development of an ultra-high sensitive immunoassay with plasma biomarker for differentiating Parkinson disease dementia from Parkinson disease using antibody functionalized magnetic nanoparticles. J Nanobiotechnol 14, 41 (2016). https://doi.org/10.1186/s12951-016-0198-5.

1.-40. (canceled)

41. A device for detecting neurotoxic amyloid-type protein aggregates, comprising a doxycycline derivative capable of binding to neurotoxic amyloid-type protein aggregates, said doxycycline derivative being immobilized on a surface, wherein the device comprises a doxycycline derivative of formula (I): 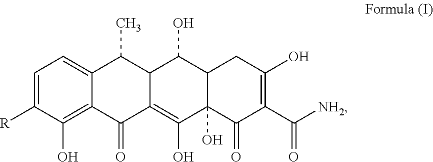
immobilized on a surface, wherein R is selected from the group consisting of:
i—a substituent selected from the group consisting of H, NH₂, COOH, (CH₂)ₙX, COOCH₂(CH₂)ₙX, wherein n=0-10 and X is selected from Cl, Br and I;
ii—an alkyl or aryl linker with a thiol end group;
iii— 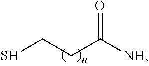
wherein n=0-10;
iv— 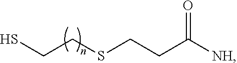
wherein n=0-10; and
v— 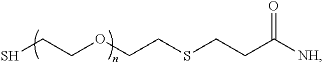
wherein n=0-10.

42. The device of claim 41, wherein R is a substituent selected from the group consisting of H, NH₂, COOH, (CH₂)ₙX, COOCH₂(CH₂)ₙX, wherein n=0-10 and X is selected from Cl, Br and I.

43. The device of claim 41, wherein the device comprises a doxycycline derivative of formula (II):

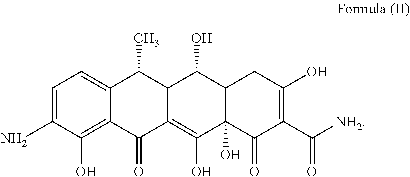 [SMILES: C[C@H]1c2ccc(N)c(O)c2C(=O)C2=C(O)[C@]3(O)C(=O)C(C(N)=O)=C(O)CC3[C@@H](O)C21]

44. The device of claim 41, wherein the surface is a polymeric surface selected from the group consisting of polystyrene, a polystyrene/divinylbenzene copolymer, and other synthetic or natural polymers where doxycycline and derivatives thereof may be immobilized.

45. The device of claim 44, wherein the polymeric surface comprises polystyrene.

46. The device of claim 44, wherein the doxycycline derivative is immobilized on the polymeric surface by being covalently bound through a linker to a blocking agent adsorbed on the surface, wherein the blocking agent is bovine serum albumin (BSA) and the linker is glutaraldehyde.

47. The device of claim 41, wherein the device comprises a doxycycline derivative of formula (II) 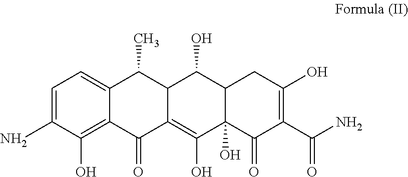 [SMILES: C[C@H]1c2ccc(N)c(O)c2C(=O)C2=C(O)[C@]3(O)C(=O)C(C(N)=O)=C(O)CC3[C@@H](O)C21]
immobilized on a polymeric surface, wherein the polymeric surface comprises polystyrene.

48. The device of claim 47, wherein the device comprises the doxycycline derivative of formula (II) immobilized on the polymeric surface by being covalently bound through glutaraldehyde as a linker to bovine serum albumin adsorbed on the surface.

49. The device of claim 41, wherein the surface is a functionalized metallic surface.

50. The device of claim 49, wherein the functionalized metallic surface is a functionalized gold surface, wherein the functionalized gold surface comprises a self-assembled monolayer (SAM) of a mercapto acid.

51. The device of claim 41, wherein the device comprises a doxycycline derivative of formula (II) 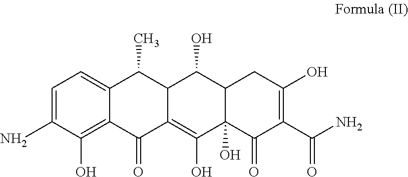 [SMILES: C[C@H]1c2ccc(N)c(O)c2C(=O)C2=C(O)[C@]3(O)C(=O)C(C(N)=O)=C(O)CC3[C@@H](O)C21]
immobilized on a functionalized metallic surface, wherein the functionalized metallic surface is a functionalized gold surface, wherein the functionalized gold surface comprises a SAM of 3-mercaptopropionic acid, and wherein the doxycycline derivative of formula (II) is covalently bound to the SAM of 3-mercaptopropionic acid.

52. An in vitro method for detecting and/or quantifying neurotoxic amyloid-type protein aggregates, comprising:
i—providing a device for detecting neurotoxic amyloid-type protein aggregates according to claim 41;
ii—contacting the device with a sample in which the presence of said aggregates is to be determined; and
iii—determining the presence and/or concentration of said aggregates by a detection technique.

53. The method of claim 52, wherein the detection technique is selected from the group consisting of an immunochemical assay and an electrochemical assay.

54. The method of claim 53, wherein the detection technique is an electrochemical assay, wherein the electrochemical assay uses a technique selected from the group consisting of cyclic voltammetry (CV) and electrochemical impedance spectroscopy (EIS) for detecting the neurotoxic amyloid-type protein aggregates.

55. The method of claim 54, wherein the electrochemical assay uses cyclic voltammetry for detecting the neurotoxic amyloid-type protein aggregates.

56. The method of claim 52, wherein the detection technique is an immunochemical assay, and wherein step iii—comprises the sub-steps of:
a) contacting the device with an antibody appropriate for binding to the neurotoxic amyloid-type protein aggregates to be detected and/or quantified; and
b) measuring the bound antibody by a measuring technique.

57. The method of claim 56, wherein the device for detecting neurotoxic amyloid-type protein aggregates comprises a doxycycline derivative immobilized on a polymeric surface by being covalently bound through a linker to a blocking agent adsorbed on the surface.

58. The method of claim 52, wherein the doxycycline derivative is the compound of formula (II).

59. A method for preparing a device for detecting neurotoxic amyloid-type protein aggregates according to claim 41, comprising:
i—providing a surface capable of immobilizing the doxycycline derivative; and
ii—contacting the surface capable of immobilizing the doxycycline derivative with a solution comprising the doxycycline derivative in conditions such that the doxycycline derivative is immobilized on the surface.

60. The method of claim 59, wherein the surface is a polymeric surface comprising a polymer selected from the group consisting of polystyrene, a polystyrene/divinylbenzene copolymer, and other synthetic or natural polymers where doxycycline and derivatives thereof may be immobilized, and wherein step i of the method further comprises the following sub-steps:
a) contacting the polymeric surface with a solution comprising a blocking agent so as to cause the adsorption of said blocking agent onto the polymeric surface, thus obtaining a blocked polymeric surface; and
b) contacting the blocked polymeric surface with a solution comprising a linker to obtain a polymeric surface capable of immobilizing a doxycycline derivative.

61. The method of claim 59, wherein the surface is a functionalized metallic surface, and the method further comprises a step prior to step i, wherein the metallic surface is contacted with a solution comprising a functionalizing agent to obtain said functionalized metallic surface.

62. The method of claim 61, wherein the metallic surface is a gold surface and the functionalizing agent is a mercapto acid.
